(19) United States
(12) Patent Application Publication
(10) Pub. No.: US 2023/0152084 A1
(43) Pub. Date: May 18, 2023

(54) Height Measurement Method and Apparatus, and Terminal

(71) Applicant: Huawei Technologies Co., Ltd., Shenzhen (CN)

(72) Inventors: Wei Fang, Shenzhen (CN); Qi Su, Shenzhen (CN); Yafei Wu, Shenzhen (CN)

(21) Appl. No.: 18/154,508

(22) Filed: Jan. 13, 2023

Related U.S. Application Data
(63) Continuation of application No. PCT/CN2021/073455, filed on Jan. 23, 2021.

(30) Foreign Application Priority Data
Jul. 15, 2020 (CN) 202010679662.1

(51) Int. Cl. G01B 11/02; G06T 17/00; G06T 7/60; G06T 7/70; G06V 10/74; G06V 40/16

(52) U.S. Cl. CPC G01B 11/022; G06T 17/00; G06T 7/60; G06T 7/70; G06V 10/761; G06V 40/161; G06T 2207/20044; G06T 2207/30201

(57) ABSTRACT

A height measurement method includes obtaining an image including a target object and a pose of a camera used when the image is photographed, obtaining pixel coordinates of at least two key skeleton points of the target object in the image, obtaining three-dimensional coordinates of the key skeleton points based on the pose of the camera and the pixel coordinates of the key skeleton points, and determining height data of the target object based on the three-dimensional coordinates of the at least two key skeleton points.

### CROSS-REFERENCE TO RELATED APPLICATIONS

This is a continuation of International Patent Application No. PCT/CN2021/073455 filed on Jan. 23, 2021, which claims priority to Chinese Patent Application No. 202010679662.1 filed on Jul. 15, 2020. The disclosures of the aforementioned applications are hereby incorporated by reference in their entireties.

### TECHNICAL FIELD

This application relates to the field of image processing technologies, and in particular, to a height measurement method and apparatus, and a terminal.

### BACKGROUND

Height is an important part of basic data of a human body and has always attracted much attention. How to quickly and accurately obtain height data of a measured object and how to obtain height data of a plurality of measured objects at the same time have always been hot topics of exploration in related fields.

In a conventional height measurement method, a measured object is required to be in a standing posture. Height data is obtained by using a standard scale or infrared or ultrasonic reflection, and the height data can only be measured one by one. In addition, a posture requirement is strict. If the standing posture is not standard, the height data is inaccurate.

In an existing height measurement method, a height of a measured object may be obtained by proportionally scaling a reference object. For example, as shown in FIG. 1, a virtual ruler is displayed in a preview interface of a terminal, and a distance between the terminal device and the measured object is obtained by using a distance sensor of the terminal. The height of the measured object is estimated based on a preset correspondence between a distance value and the ruler.

Because a measurement result of the height measurement method is obtained by using the virtual ruler to proportionally scale up the distance between the terminal and the measured object, both resolution of the terminal device and accuracy of the distance sensor of the terminal device affect measurement accuracy, and accuracy of the height measurement result is low when the resolution is insufficient or an ambient environment of the measured object is disordered.

### SUMMARY

Embodiments of this application provide a height measurement method, to measure a height of a target object, so that accuracy of a measurement result can be improved.

A first aspect of embodiments of this application provides a height measurement method including obtaining an image including a target object and a pose of a camera used when the image is photographed; obtaining pixel coordinates of at least two key skeleton points of the target object in the image, where the key skeleton point includes a skeleton joint, and the pixel coordinates indicate two-dimensional location information of the key skeleton points in the image; obtaining three-dimensional coordinates of the at least two key skeleton points based on the pose of the camera and the pixel coordinates of the at least two key skeleton points, where the three-dimensional coordinates indicate three-dimensional location information of the key skeleton points in a coordinate system, and the three-dimensional coordinates of the at least two key skeleton points indicate information about a distance between the at least two key skeleton points; and determining height data of the target object based on the three-dimensional coordinates of the at least two key skeleton points.

According to the height measurement method provided in this embodiment of this application, a two-dimensional image obtained by photographing the target object may be detected according to a skeleton detection algorithm or the like. The pixel coordinates of the key skeleton point in the image may be obtained. The pixel coordinates of the key skeleton point may be converted into three-dimensional coordinates in three-dimensional space based on the pose that is of the camera and that corresponds to the two-dimensional image. The three-dimensional coordinates correspond to location information of the key skeleton point in the real world. Therefore, the height data of the target object may be directly obtained. According to the height measurement method provided in this solution, the height data of the target object may be obtained in a contactless manner based on the photographed two-dimensional image of the target object. In addition, there is no need for a height reference object in a photographing scenario. This can reduce an error and improve measurement accuracy.

In a possible implementation of the first aspect, the determining height data of the target object based on the three-dimensional coordinates of the at least two key skeleton points further includes obtaining pixel coordinates of at least three key skeleton points of the target object in the image; obtaining three-dimensional coordinates of the at least three key skeleton points based on the pose of the camera and the pixel coordinates of the at least three key skeleton points, where the three-dimensional coordinates indicate three-dimensional location information of the key skeleton points in a coordinate system, and the three-dimensional coordinates of the at least three key skeleton points indicate information about distances between the at least three key skeleton points; and determining at least two skeleton distances based on the three-dimensional coordinates of the at least three key skeleton points, and determining the height data of the target object based on the at least two skeleton distances.

In a possible implementation of the first aspect, the coordinate system includes a world coordinate system.

In a possible implementation of the first aspect, the method further includes obtaining three-dimensional point cloud information of the target object; and obtaining three-dimensional coordinates of the at least two key skeleton points of the target object based on the pose of the camera and the pixel coordinates of the key skeleton points further includes obtaining, based on the pixel coordinates of the key skeleton points, the pose of the camera, and the three-dimensional point cloud information, the three-dimensional coordinates of the at least two key skeleton points according to an impact detection algorithm.

This method provides a specific solution for converting the pixel coordinates of the key skeleton point into the three-dimensional coordinates of the key skeleton point, that is, from two-dimensional information to three-dimensional information. The three-dimensional coordinates of the key skeleton point are obtained based on the three-dimensional point cloud information and the impact detection algorithm. Therefore, compared with direct calculation performed based on the pose of the camera, this method can improve accuracy of the three-dimensional coordinates.

In a possible implementation of the first aspect, obtaining the three-dimensional point cloud information of the target object further includes obtaining the three-dimensional point cloud information of the target object based on at least two images of the target object photographed from different orientations.

This method provides a specific method for obtaining the three-dimensional point cloud information. To be specific, the three-dimensional point cloud information of the target object may be obtained by obtaining a plurality of images of the target object, and detecting and matching feature points of a plurality of images of the target object. Because the three-dimensional point cloud information is obtained based on information about the plurality of images, richer information is included in the three-dimensional point cloud information compared with a single image. This can improve accuracy of the three-dimensional coordinates.

In a possible implementation of the first aspect, obtaining three-dimensional point cloud information of the target object further includes obtaining the three-dimensional point cloud information that is of the target object and that is collected by a depth sensor. The depth sensor includes a binocular camera, a laser radar, a millimeter-wave radar, or a time of flight (TOF) sensor.

This method provides another method for obtaining the three-dimensional point cloud information. The three-dimensional point cloud information is collected by using the depth sensor. Because a three-dimensional point cloud obtained by using the depth sensor may be a dense point cloud, richer information can be included. Based on the dense three-dimensional point cloud, the obtained three-dimensional coordinates of the key skeleton point are more accurate.

In a possible implementation of the first aspect, obtaining an image of a target object and a pose of a camera used when the image is photographed further includes obtaining the at least two images of the target object photographed from different orientations, where the at least two images of the target object photographed from different orientations include the image; and obtaining the pose of the camera based on the at least two images of the target object photographed from different orientations.

This method provides a further manner of obtaining the pose of the camera. To be specific, the pose of the camera used when the image is photographed may be estimated by obtaining the at least two images of the target object photographed from different orientations, and detecting and matching the feature points.

In a possible implementation of the first aspect, obtaining an image of a target object and a pose of a camera used when the image is photographed further includes obtaining the at least two images of the target object photographed from different orientations, where the at least two images of the target object photographed from different orientations include the image of the target object; obtaining inertial measurement unit data that is of the camera and that corresponds to the at least two images of the target object photographed from different orientations; and determining the pose of the camera based on the inertial measurement unit data and the at least two images of the target object photographed from different orientations.

This method provides a further manner of obtaining the pose of the camera. In addition to obtaining the at least two images of the target object photographed from different orientations, the inertial measurement unit data may be further collected. This can improve accuracy of calculating the pose of the camera.

In a possible implementation of the first aspect, determining height data of the target object based on the three-dimensional coordinates of the at least two key skeleton points further includes obtaining a skeleton length of the target object and posture information of the target object based on the three-dimensional coordinates of the at least two key skeleton points; determining a preset weight parameter of the skeleton length based on the posture information; and determining the height data of the target object based on the skeleton length and the weight parameter.

According to the height measurement method provided in this method, considering that the three-dimensional coordinates of the key skeleton point come from a body surface of the target object, there is a specific error between an actual height corresponding to a skeleton in a body and the skeleton length obtained based on the three-dimensional coordinates. Therefore, the weight parameter is introduced to correct the calculated skeleton length. This can improve precision of the solution.

In a possible implementation of the first aspect, the skeleton length includes a skeleton length of a head and a skeleton length of a leg. Determining the height data of the target object based on the skeleton length and the weight parameter further includes determining a head height compensation value based on the skeleton length of the head and a preset head compensation parameter; determining a foot height compensation value based on the skeleton length of the leg and a preset foot compensation parameter; and determining the height data of the target object based on the skeleton length information, the weight parameter, the head height compensation value, and the foot height compensation value.

According to the height measurement method provided in this method, compensation of the head and the foot is introduced. This can further improve height measurement accuracy.

In a possible implementation of the first aspect, the method further includes performing face detection on the image, and obtaining head height data of the target object. The head height data is used to correct pixel coordinates that are of the key skeleton point and that correspond to the head, in two-dimensional key skeleton point information.

According to the height measurement method provided in this method, the head height data may be further obtained through face detection, and the pixel coordinates of the key skeleton point are corrected. This improves measurement accuracy.

In a possible implementation of the first aspect, the image includes at least two target objects. The method further includes performing face detection on the image, and determining pixel coordinates of a key skeleton point of each of the at least two target objects from the pixel coordinates of the key skeleton points according to an image segmentation algorithm.

According to the height measurement method provided in this method, heights of a plurality of target objects in an image can be measured. Compared with the conventional technology in which the heights are detected one by one, in this method, an operation can be simplified. This improves measurement efficiency.

In a possible implementation of the first aspect, the method further includes displaying information about the at least two target objects to a user, where the information about the at least two target objects includes at least one of image information of the at least two target objects, image information marked with pixel coordinates of key skeleton points of the at least two target objects, and face detection result information of the at least two target objects; and obtaining a user instruction. The user instruction instructs to perform height measurement on one or more of the at least two target objects.

According to the height measurement method provided in this method, interaction with the user may be further performed, and an object whose height the user wants to detect is selected, based on the user instruction, from target objects included in the image. This improves user experience.

In a possible implementation of the first aspect, the key skeleton points are arranged in a direction of gravity. The key skeleton points arranged in the direction of gravity help improve accuracy of height measurement.

In a possible implementation of the first aspect, the target object is in a non-standing posture. The non-standing posture includes a sitting posture, a lying posture, and a kneeling posture. When the target object is in the non-standing posture, a height of the target object can also be measured in this implementation of this application.

In a possible implementation of the first aspect, determining height data of the target object based on the three-dimensional coordinates of the at least two key skeleton points further includes obtaining skeleton length information of the target object based on the three-dimensional coordinates of the at least two key skeleton points; deleting skeleton length information that meets a first preset condition, where the first preset condition includes skeleton length information in which a skeleton length falls outside a preset range, or a skeleton length difference between symmetric parts being greater than or equal to a preset threshold range; and determining the height data of the target object based on skeleton length information obtained after deletion.

According to the height measurement method provided in this method, abnormal data may also be deleted. This improves accuracy of a measurement result. Optionally, based on symmetry of a human body, skeletons of left and right symmetric parts may be verified. For example, a difference between skeleton lengths corresponding to a left leg and a right leg should be small. If the difference is greater than a threshold, the abnormal data may be deleted.

In a possible implementation of the first aspect, the method further includes labeling the height data of the target object near the target object in the image, and displaying the height data to the user; or broadcasting the height data of the target object through voice.

According to the height measurement method provided in this method, the height of the target object may be marked in an image displayed in real time, and feedback is provided in real time. This improves user experience.

In a possible implementation of the first aspect, the method further includes: if the key skeleton point of the target object does not meet a second preset condition, displaying detection failure information to the user, or prompting the user with the detection failure information through voice, or prompting the user with the detection failure information through vibration.

According to the height measurement method provided in this method, when detection fails, feedback may be provided to the user. This improves user experience.

A second aspect of embodiments of this application provides a height measurement apparatus including an obtaining module configured to obtain an image including a target object and a pose of a camera used when the image is photographed, where the obtaining module is further configured to obtain pixel coordinates of at least two key skeleton points of the target object in the image, where the key skeleton point includes a skeleton joint, and the pixel coordinates indicate two-dimensional location information of the key skeleton points in the image; and the obtaining module is further configured to obtain three-dimensional coordinates of the at least two key skeleton points based on the pose of the camera and the pixel coordinates of the key skeleton points, where the three-dimensional coordinates indicate three-dimensional location information of the key skeleton points in a coordinate system, and the three-dimensional coordinates of the at least two key skeleton points indicate information about a distance between the at least two key skeleton points; and a determining module configured to determine height data of the target object based on the three-dimensional coordinates of the at least two key skeleton points.

In a possible implementation of the second aspect, the obtaining module is further configured to obtain pixel coordinates of at least three key skeleton points of the target object in the image; and obtain three-dimensional coordinates of the at least three key skeleton points based on the pose of the camera and the pixel coordinates of the at least three key skeleton points. The three-dimensional coordinates indicate three-dimensional location information of the key skeleton points in a coordinate system. The three-dimensional coordinates of the at least three key skeleton points indicate information about distances between the at least three key skeleton points.

The determining module is further configured to determine at least two skeleton distances based on the three-dimensional coordinates of the at least three key skeleton points, and determine the height data of the target object based on the at least two skeleton distances.

In a possible implementation of the second aspect, the coordinate system includes a world coordinate system.

In a possible implementation of the second aspect, the obtaining module is further configured to obtain three-dimensional point cloud information of the target object. The obtaining three-dimensional coordinates of the at least two key skeleton points of the target object based on the pose of the camera and the pixel coordinates of the key skeleton points further includes obtaining, based on the pixel coordinates of the key skeleton points, the pose of the camera, and the three-dimensional point cloud information, the three-dimensional coordinates of the at least two key skeleton points according to an impact detection algorithm.

In a possible implementation of the second aspect, the obtaining module is further configured to obtain the three-dimensional point cloud information of the target object based on at least two images of the target object photographed from different orientations.

In a possible implementation of the second aspect, the obtaining module is further configured to obtain the three-dimensional point cloud information that is of the target object and that is collected by a depth sensor. The depth sensor includes a binocular camera, a laser radar, a millimeter-wave radar, or a time of flight sensor.

In a possible implementation of the second aspect, the obtaining module is further configured to obtain the at least two images of the target object photographed from different orientations, where the at least two images of the target object photographed from different orientations include the image; and obtain the pose of the camera based on the at least two images of the target object photographed from different orientations.

In a possible implementation of the second aspect, the obtaining module is further configured to obtain the at least two images of the target object photographed from different orientations, where the at least two images of the target object photographed from different orientations include the image of the target object; obtain inertial measurement unit data that is of the camera and that corresponds to the at least two images of the target object photographed from different orientations; and determine the pose of the camera based on the inertial measurement unit data and the at least two images of the target object photographed from different orientations.

In a possible implementation of the second aspect, the determining module is further configured to obtain a skeleton length of the target object and posture information of the target object based on the three-dimensional coordinates of the at least two key skeleton points; determine a preset weight parameter of the skeleton length based on the posture information; and determine the height data of the target object based on the skeleton length and the weight parameter.

In a possible implementation of the second aspect, the skeleton length includes a skeleton length of a head and a skeleton length of a leg. The determining module is further configured to determine a head height compensation value based on the skeleton length of the head and a preset head compensation parameter; determine a foot height compensation value based on the skeleton length of the leg and a preset foot compensation parameter; and determine the height data of the target object based on the skeleton length information, the weight parameter, the head height compensation value, and the foot height compensation value.

In a possible implementation of the second aspect, the image includes at least two target objects. The apparatus further includes a processing module configured to perform face detection on the image, and determine pixel coordinates of a key skeleton point of each of the at least two target objects from the pixel coordinates of the key skeleton points according to an image segmentation algorithm.

In a possible implementation of the second aspect, the apparatus further includes an output module configured to display information about the at least two target objects to a user. The information about the at least two target objects includes at least one of image information of the at least two target objects, image information marked with pixel coordinates of key skeleton points of the at least two target objects, and face detection result information of the at least two target objects. The obtaining module is further configured to obtain a user instruction. The user instruction instructs to perform height measurement on one or more of the at least two target objects.

In a possible implementation of the second aspect, the key skeleton points are arranged in a direction of gravity. The key skeleton points arranged in the direction of gravity help improve accuracy of height measurement.

In a possible implementation of the second aspect, the target object is in a non-standing posture. The non-standing posture includes a sitting posture, a lying posture, and a kneeling posture. When the target object is in the non-standing posture, a height of the target object can also be measured in this implementation of this application.

In a possible implementation of the second aspect, the determining module is further configured to obtain skeleton length information of the target object based on the three-dimensional coordinates of the at least two key skeleton points; delete skeleton length information that meets a first preset condition, where the first preset condition includes skeleton length information in which a skeleton length falls outside a preset range, or a skeleton length difference between symmetric parts being greater than or equal to a preset threshold range; and determine the height data of the target object based on skeleton length information obtained after deletion.

In a possible implementation of the second aspect, the apparatus further includes an output module configured to label the height data of the target object near the target object in the image, and display the height data to the user; or broadcast the height data of the target object through voice.

In a possible implementation of the second aspect, the apparatus further includes an output module configured to: if the key skeleton point of the target object does not meet a second preset condition, display detection failure information to the user, or prompt the user with the detection failure information through voice, or prompt the user with the detection failure information through vibration.

A third aspect of embodiments of this application provides a terminal including one or more processors and a memory. The memory stores computer-readable instructions. The one or more processors read the computer-readable instructions in the memory such that the terminal implements the method according to any one of the first aspect and the possible implementations.

A fourth aspect of embodiments of this application provides a computer program product including instructions. When the computer program product runs on a computer, the computer is enabled to perform the method according to any one of the first aspect and the possible implementations.

A fifth aspect of embodiments of this application provides a computer-readable storage medium including instructions. When the instructions are run on a computer, the computer is enabled to perform the method according to any one of the first aspect and the possible implementations.

A sixth aspect of embodiments of this application provides a chip including a processor. The processor is configured to read and execute a computer program stored in a memory to perform the method according to any possible implementation of any one of the foregoing aspects. Optionally, the chip includes a memory, and the memory and the processor are connected by using a circuit or a wire. Further, optionally, the chip further includes a communication interface, and the processor is connected to the communication interface. The communication interface is configured to receive data and/or information that needs to be processed. The processor obtains the data and/or the information from the communication interface, processes the data and/or the information, and outputs a processing result through the communication interface. The communication interface may be an input/output interface.

For technical effect brought by any one of the implementations of the second aspect to the sixth aspect, refer to the technical effects brought by corresponding implementations in the first aspect. Details are not described herein again.

It can be learned from the foregoing technical solutions that embodiments of this application have the following advantages:

According to the height measurement method provided in this embodiment of this application, the image of the target object and the pose of the camera used when the image is photographed are obtained, skeleton detection may be performed on the image to obtain the pixel coordinates of the at least two key skeleton points of the target object in the image, then the pixel coordinates of the key skeleton points are converted into three-dimensional space based on the pose of the camera to obtain the three-dimensional coordinates of the at least two key skeleton points, and finally the height data of the target object is determined based on the three-dimensional coordinates of the at least two key skeleton points. In this method, the two-dimensional pixel coordinates of the key skeleton points are converted into the three-dimensional coordinates, and the height data of the target object is directly obtained without conversion of a reference object. This can avoid a measurement error caused by conversion of the reference object when a scenario around the target object is complex, and can improve accuracy of a height measurement result.

In addition, regardless of a posture of the target object, skeleton information indicated by the key skeleton point of the target object does not change. Therefore, the height measurement method provided in this embodiment of this application may be applied to height measurement of the target object in various postures.

### BRIEF DESCRIPTION OF DRAWINGS

FIG. 1 is a schematic diagram of an embodiment of height measurement;

FIG. 2A is a schematic diagram of an embodiment of an application scenario of a height measurement method according to an embodiment of this application;

FIG. 2B is a schematic diagram of an embodiment of an application scenario of a height measurement method according to an embodiment of this application;

FIG. 3 is a schematic diagram of an embodiment of a height measurement method according to an embodiment of this application;

FIG. 4 is a schematic diagram of another embodiment of a height measurement method according to an embodiment of this application;

FIG. 5 is a schematic diagram of converting a two-dimensional key skeleton point into a three-dimensional key skeleton point according to an embodiment of this application;

FIG. 6 is a schematic diagram of a height measurement method in a standing posture according to an embodiment of this application;

FIG. 7 is a schematic diagram of a height measurement method in a sitting posture according to an embodiment of this application;

FIG. 8 is a schematic diagram of an application scenario of a height measurement method according to an embodiment of this application;

FIG. 9A is a schematic diagram of a point cloud of a simultaneous localization and mapping (SLAM) system according to an embodiment of this application;

FIG. 9B is a schematic diagram of a two-dimensional key skeleton point detection result according to an embodiment of this application;

FIG. 9C is a schematic diagram of height detection during measurement at different angles according to an embodiment of this application;

FIG. 10 is a schematic diagram of an embodiment of a height measurement apparatus according to an embodiment of this application;

FIG. 11A is a schematic diagram of another embodiment of a height measurement apparatus according to an embodiment of this application;

FIG. 11B is a schematic diagram of another embodiment of a height measurement apparatus according to an embodiment of this application; and

FIG. 12 is a schematic diagram of an embodiment of a terminal according to an embodiment of this application.

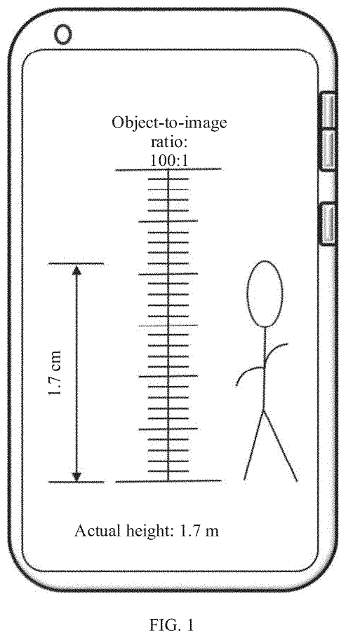

### DESCRIPTION OF EMBODIMENTS

Embodiments of this application provide a height measurement method to measure a height of a target object in a plurality of postures such that accuracy of height data can be improved.

For ease of understanding, the following briefly describes some technical terms in embodiments of this application.

1. Human key skeleton point detection: Pose estimation, which mainly detects some key points of a human body, such as joints and facial features, and provides skeleton information based on the key points. Key skeleton points are also referred to as skeleton joints or joints.

2. Intrinsic and extrinsic camera parameters.

The intrinsic camera parameter is a parameter related to characteristics of a camera itself, and includes a focal length, a pixel size, and the like of the camera. For configuration of an electronic device provided with a camera, the intrinsic camera parameter is usually known.

The extrinsic camera parameter is a parameter in a world coordinate system, and includes a location and a rotation direction of the camera.

It may be determined, based on the intrinsic camera parameter and the extrinsic camera parameter, that two-dimensional pixels in an image photographed by the camera correspond to three-dimensional coordinates in the world coordinate system.

3. Pose of a camera.

A location and a posture of a camera in a world coordinate system when the camera photographs an image are known, and an extrinsic camera parameter may be obtained based on the known pose of the camera. The pose of the camera includes six degrees of freedom (DoF). Three DoF related to the location are used to determine the location of the camera in three-dimensional space. Three DoF related to a rotation angle are used to determine a rotation posture of the camera in the three-dimensional space. The pose of the camera corresponds to the location and the posture of the camera in the world coordinate system when the image is photographed. For an image sequence that is obtained through continuous photographing and that is used to calculate the pose of the camera, relative movement is required between the camera and a photographed object, and includes relative location and posture changes. Further, the photographed object may be stationary and the camera may move. Alternatively, the photographed object may move and the camera may be stationary. Alternatively, both the photographed object and the camera may move, and there is a relative pose change between the photographed object and the camera.

The following describes embodiments of this application with reference to the accompanying drawings. It is clear that the described embodiments are merely some but not all of embodiments of this application. A person of ordinary skill in the art may learn that, with development of technologies and emergence of a new scenario, the technical solutions provided in embodiments of this application are also applicable to a similar technical problem.

In this specification, claims, and accompanying drawings of this application, the terms “first”, “second”, and so on are intended to distinguish between similar objects but do not necessarily indicate a specific order or sequence. It should be understood that data termed in such a way is interchangeable in proper circumstances such that embodiments described herein can be implemented in other orders than the order illustrated or described herein. In addition, the terms “include”, “contain” and any other variants mean to cover the non-exclusive inclusion, for example, a process, method, system, product, or device that includes a list of steps or modules is not necessarily limited to those steps or modules, but may include other steps or modules not expressly listed or inherent to such a process, method, product, or device. Naming or numbering of steps in this application does not mean that the steps in the method procedures need to be performed in a time/logical order indicated by the naming or numbering. An execution order of the steps in the procedures that have been named or numbered can be changed based on a technical objective to be achieved, as long as same or similar technical effects can be achieved.

According to the height measurement method provided in embodiments of this application, a measured target object may be a vertebrate. Further, a person is used as an example for description in embodiments of this application.

The height measurement method provided in embodiments of this application is applicable to a plurality of height measurement scenarios. The following uses examples for description.

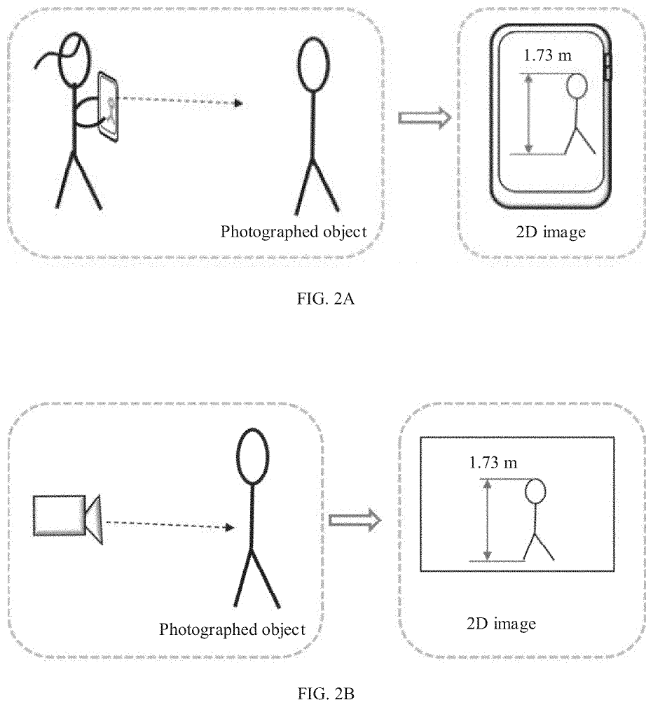

Scenario 1: In an application of an augmented reality (AR) technology or a virtual reality (VR) technology, height measurement may be performed by using an intelligent terminal device. For example, as shown in FIG. 2A, a smartphone is used to scan an ambient environment of a measured object (or referred to as a target object, a measured object, or the like, which may be briefly referred to as an object). A SLAM system is used to estimate a pose of a camera and obtain three-dimensional (3D) point cloud data of the ambient environment of the measured object. A two-dimensional (2D) key skeleton point in an image, that is, pixel coordinates of the key skeleton point, is obtained according to a skeleton detection algorithm and a face recognition algorithm. Three-dimensional coordinates of at least two key skeleton points in three-dimensional space are obtained based on the 3D point cloud data. Data integration is performed on three-dimensional information of the key skeleton point to output height data of one or more measured objects, to measure heights of a plurality of users in a plurality of postures. The height data may also be superimposed near the measured object in the image, and output on a display of the smartphone. In the following embodiments, the height measurement method is described by using the scenario 1 as an example.

Scenario 2: As shown in FIG. 2B, an image capture device is fastened. An image is captured when a measured object walks through a predetermined location. Because a location of a camera in a world coordinate system is known, skeleton detection is performed on the photographed image to obtain a 2D key skeleton point, and after the 2D key skeleton point is converted into a 3D key skeleton point, height data of the measured object can be output through data integration and calculation.

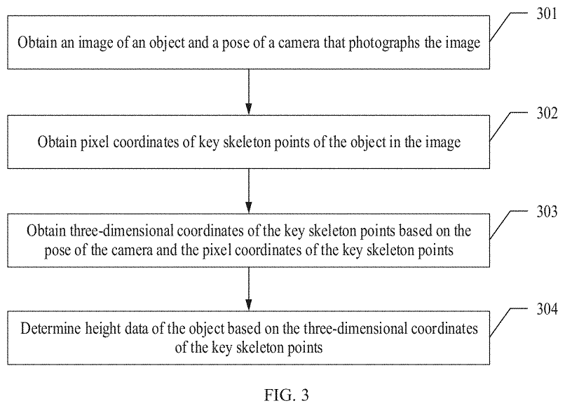

The following describes a height measurement method in detail. FIG. 3 is a schematic diagram of an embodiment of a height measurement method according to an embodiment of this application.

301: Obtain an image of an object and a pose of a camera that photographs the image.

In this application, a height measurement apparatus may be a terminal. The terminal may obtain the image of the object by using an image capture apparatus such as a camera. The camera may be provided with a common monocular camera or a binocular camera. This is not specifically limited herein. The camera may be a component built in the terminal, or may be a device outside the terminal. Image data may be transmitted to the terminal through a communication connection. It should be noted that an intrinsic camera parameter is known.

The terminal further obtains the pose that is of the camera and that corresponds to the image. Optionally, the terminal photographs the object from different orientations by using the monocular camera, to obtain at least two images of the object, and calculates the pose of the camera by detecting homologous feature point pairs in the images. Alternatively, the terminal photographs the object by using the binocular camera to obtain the pose of the camera. An inertial measurement unit (IMU) is an apparatus for measuring a three-axis attitude angle (or angular rate) and an acceleration of an object. Optionally, if the terminal includes the IMU and the camera configured to capture the image of the object, the pose of the camera may be obtained based on IMU data used in an image capture process of the camera. Optionally, the pose of the camera is calculated based on the at least two captured images of the object and the IMU data used when the images are captured. It may be understood that the pose of the camera obtained based on a plurality of images of the object and the IMU data is more accurate.

Optionally, the image of the object may include one or more to-be-measured objects.

302: Obtain pixel coordinates of key skeleton points of the object in the image.

The key skeleton point includes a skeleton joint. Key skeleton point recognition may be performed on the image according to various existing skeleton detection algorithms, to obtain pixel coordinates of at least two key skeleton points of the target in the image. The pixel coordinates may indicate two-dimensional location information of the key skeleton points in the image. The pixel coordinates (u, v) indicate location of the key skeleton point in the image.

The key skeleton point may be detected according to the skeleton detection algorithm. Further, there are a plurality of key skeleton point detection algorithms, for example, a regional multi-person pose estimation (RMPE) algorithm and a DeepCut algorithm. A quantity of key skeleton points may be, for example, 14 or 21.

Optionally, if the image of the object includes a plurality of to-be-measured objects, two-dimensional key skeleton point information of each to-be-measured object may be separately obtained. The two-dimensional key skeleton point information includes pixel coordinates of each key skeleton point in the image, and further includes an identifier of each key skeleton point.

Optionally, the target object may be in a standing posture. The standing posture means that all key skeleton points of the target object are arranged along a direction of gravity, or arranged vertically in the posture. The key skeleton points arranged in the direction of gravity or arranged vertically help improve accuracy of height measurement.

Optionally, the target object may be in a non-standing posture. The non-standing posture means that pixel coordinates of some key skeleton points of the target object are not arranged in a direction of gravity or arranged vertically in the posture, that is, not all pixel coordinates of key skeleton points in the non-standing posture are arranged in a vertical straight line. The non-standing posture includes a sitting posture, a lying posture, a kneeling posture, or another posture. In this solution, when the target object is in the non-standing posture, a height can also be measured.

303: Obtain three-dimensional coordinates of the key skeleton points based on the pose of the camera and the pixel coordinates of the key skeleton point.

Because the intrinsic camera parameter is known, pixel coordinates of the two-dimensional key skeleton points in the image may be converted into three-dimensional coordinates in a world coordinate system based on the pose of the camera, to obtain three-dimensional coordinates of at least two key skeleton points. The three-dimensional coordinates indicate three-dimensional location information of the key skeleton points in the world coordinate system. The three-dimensional coordinates are, for example, (x, y, z). In addition, because the three-dimensional coordinates of the at least two key skeleton points are obtained, to distinguish between different key skeleton points, an identifier of each key skeleton point may be further obtained.

The three-dimensional coordinates of the at least two key skeleton points may indicate information about a distance between the at least two key skeleton points. For example, three-dimensional coordinates of a first key skeleton point are (x1, y1, z1), and three-dimensional coordinates of a second key skeleton point are (x2, y2, z2), a distance between the first key skeleton point and the second key skeleton point in the world coordinate system may be calculated. It may be understood that if the first key skeleton point and the second key skeleton point are two endpoints of a same skeleton, that is, associated key skeleton points, a skeleton length may be calculated based on three-dimensional coordinates of the two key skeleton points. In other words, the information about the distance between the at least two key skeleton points includes skeleton length information such that the information can be used to calculate a height of the object.

304: Determine height data of the object based on the three-dimensional coordinates of the key skeleton points.

The skeleton length may be obtained based on the three-dimensional coordinates of the at least two key skeleton points. Further, a skeleton length may be calculated based on three-dimensional coordinates of two associated key skeleton points. Optionally, at least two skeleton distances are determined based on three-dimensional coordinates of at least three key skeleton points, and a skeleton structure of the object is used to perform splicing calculation based on the skeleton length information such that the height data of the object may be obtained based on the at least two skeleton distances. For example, the skeleton length may be calculated based on a three-dimensional space Euclidean distance between 3D coordinates of two joints that form the skeleton.

To obtain the height data of the object, a plurality of skeleton lengths is usually required. To distinguish between different skeleton length information, an identifier of a skeleton corresponding to each skeleton length may be further obtained. The identifier of the skeleton may be a human torso type (for example, “arm” or “leg”) corresponding to the skeleton, and indicates different skeletons. There is a correspondence between an identifier of a skeleton and an identifier of a key skeleton point. For example, a key skeleton point identified as a right shoulder and a key skeleton point identified as a right elbow may jointly form a skeleton identified as a right upper arm.

A skeleton splicing algorithm is used to obtain the height data based on the skeleton length. There is a plurality of specific calculation methods, which are not limited herein.

According to the height measurement method provided in this embodiment of this application, pixel coordinates of the key skeleton points of the object in the image are detected, then the pixel coordinates of the key skeleton points are converted into three-dimensional space based on the pose of the camera, to obtain three-dimensional coordinates of the key skeleton point, and the height data of the object is finally determined based on the three-dimensional coordinates of the at least two key skeleton points. In this method, the two-dimensional pixel coordinates of the key skeleton points are converted into the three-dimensional coordinates, and the height data of the object is directly obtained without conversion of a reference object. This can avoid a measurement error caused by conversion of the reference object when a scenario around the object is complex, and can improve accuracy of a height measurement result.

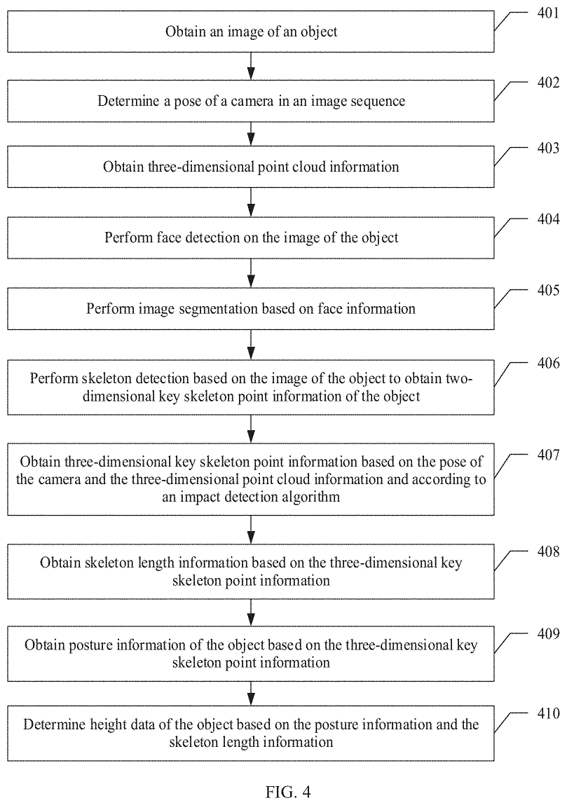

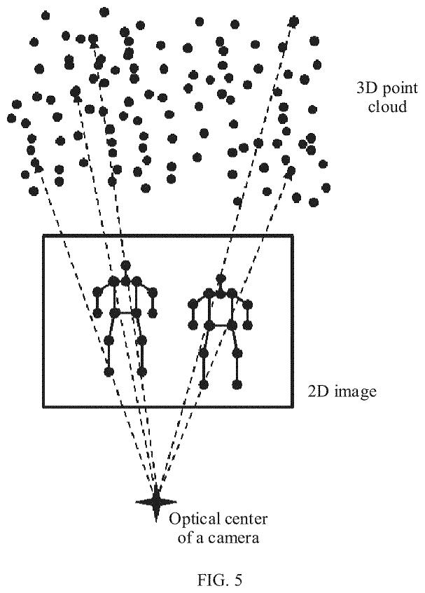

FIG. 4 is a schematic diagram of another embodiment of a height measurement method according to an embodiment of this application.

401: Obtain an image of an object.

The terminal obtains at least two images of the object. The at least two images of the object are photographed by using a camera in different poses.

Optionally, IMU data used when the at least two images of the object are photographed may be simultaneously obtained. Because poses of the camera are different when the at least two images of the object are captured, the IMU data may indicate a movement direction and a movement distance of the camera.

It should be noted that the image may include one or more objects whose heights are to be measured. For each object, at least two images of the object need to be obtained to perform height measurement.

402: Determine a pose of the camera in an image sequence.

The pose of the camera may be calculated based on the at least two images of the object by detecting homologous feature point pairs in the images. Alternatively, the pose of the camera is obtained based on the IMU data used in an image capture process of the camera. Alternatively, the pose of the camera is calculated based on the at least two images of the object and the IMU data used when the images are captured. It may be understood that the pose of the camera obtained based on a plurality of images of the object and the IMU data is more accurate.

The terminal may obtain a pose that is of the camera and that corresponds to any one of the at least two images of the target.

403: Obtain three-dimensional point cloud information.

The terminal obtains three-dimensional point cloud information. The three-dimensional point cloud information includes three-dimensional coordinates of a visible part of the object in a coordinate system. Optionally, the coordinate system includes a world coordinate system. Optionally, a method for obtaining the three-dimensional point cloud information includes a lidar depth imaging method, a computer stereo vision imaging method, a structured light method, or the like. This is not specifically limited herein.

For example, the three-dimensional point cloud information is obtained by using the computer stereo vision imaging method. To be specific, feature extraction and matching are performed on the at least two images that are of the object and that are obtained in the step 401 to obtain a feature point pair. A three-dimensional point cloud corresponding to a pixel in the image of the object is obtained according to a triangulation algorithm and based on the feature point pair and the pose of the camera determined in the step 402.

For example, the three-dimensional point cloud information is obtained by using the lidar depth imaging method. If the terminal includes a depth sensor, for example, a laser sensor, the 3D point cloud information may be directly obtained. Based on a specific configuration of the depth sensor, the output 3D point cloud information may be a dense 3D point cloud or a semi-dense 3D point cloud.

Optionally, the 3D point cloud information may be obtained in combination with the foregoing two manners. That is, when the 3D point cloud is calculated based on the images of the object and the pose of the camera, a point cloud depth is directly provided by a depth map obtained by the depth sensor. In this way, accuracy of the 3D point cloud can be improved. In addition, the pose of the camera can also be optimized such that the pose of the camera is more accurate.

404: Perform face detection on the image of the object.

The image of the object may include one or more objects whose heights are to be measured. Face information of the one or more to-be-measured objects may be determined by performing face detection on the image of the object.

Optionally, if the image of the object includes a plurality of pieces of face information, the terminal may further present a face detection result to a user, for example, present face information of each object on a display, or output a quantity of objects through voice.

405: Perform image segmentation based on the face information.

The face information of the one or more to-be-measured objects may be determined based on the face detection result in the step 404.

If the image of the object includes a plurality of pieces of face information, image segmentation may be performed on the image of the object to obtain image parts of a plurality of to-be-measured objects. The image parts of the plurality of to-be-measured objects may be respectively used to measure heights of the plurality of to-be-measured objects.

It should be noted that the step 404 and the step 405 are optional, and may be performed or not performed. This is not limited herein.

406: Perform skeleton detection based on the image of the object to obtain two-dimensional key skeleton point information of the object.

Two-dimensional key skeleton point information of the image of the object is obtained according to a key skeleton point detection algorithm. The two-dimensional key skeleton point information herein includes pixel coordinates of a key skeleton point and an identifier that is of the key skeleton point and that corresponds to the pixel coordinates.

A human key skeleton point may be detected according to a skeleton detection algorithm. Further, there are a plurality of key skeleton point detection algorithms. For example, a quantity of human key skeleton points may be 14 or 21. For example, 14 points are used as an example. Table 1 shows meanings and numbers of human key skeleton points. Pixel coordinates of each human key skeleton point in the image may be output according to the skeleton detection algorithm, and are identified by a preset number.

TABLE 1
Human key skeleton points

| | | | | |
|---|---|---|---|---|
| 1/Right shoulder | 2/Right elbow | 3/Right wrist | 4/Left shoulder | 5/Left elbow |
| 6/Left wrist | 7/Right hip | 8/Right knee | 9/Right ankle | 10/Left hip |
| 11/Left knee | 12/Left ankle | 13/Top of a head | 14/Neck | — |

Optionally, if the image includes a plurality of to-be-measured objects, two-dimensional key skeleton point information of each to-be-measured object may be obtained according to the key skeleton point detection algorithm. Optionally, if the step 404 is performed, skeleton detection is performed on the image of the object to obtain human key skeleton points of all to-be-measured objects in the image, and then two-dimensional key skeleton point information corresponding to a face detection result of each to-be-measured object is determined. Alternatively, skeleton detection is separately performed on the images determined through image segmentation in the step 405, to obtain two-dimensional key skeleton point information corresponding to each to-be-measured object.

Optionally, if the image includes a plurality of to-be-measured objects, information about all to-be-measured objects is displayed to the user. The information about the to-be-measured objects includes at least one of image information of the to-be-measured objects, two-dimensional key skeleton point information of the to-be-measured objects, and face detection result information of the to-be-measured objects. Then, a user instruction is obtained, and one or more of at least two to-be-measured objects are determined as objects for height measurement based on the user instruction.

Optionally, the two-dimensional key skeleton point information of the object is verified based on the face detection result. Further, in the two-dimensional key skeleton point information, a key skeleton point corresponding to a head is usually a single joint, and face information recognized in face detection may indicate jaw-to-hairline information. Therefore, pixel coordinates of the two-dimensional key skeleton point corresponding to the head may be verified based on the face detection result. This can improve accuracy of a height measurement result in this solution.

Optionally, if two-dimensional key skeleton point information of an object does not meet a second preset condition, the detection failure information is displayed to the user, or the user is prompted with the detection failure information through voice, or the user is prompted with the detection failure information through vibration. This is not further limited herein.

Optionally, the second preset condition may be that no key skeleton point is detected. Alternatively, the second preset condition may be that a quantity of key skeleton points is less than or equal to a preset threshold, for example, 5, 6, or 7. Alternatively, the second preset condition is that a quantity of skeletons indicated by the detected key skeleton point is less than or equal to a preset threshold, for example, 3 or 4. Alternatively, the second preset condition is that a skeleton type and a quantity that are indicated by the detected key skeleton point do not meet a preset requirement. For example, the skeleton type indicated by the key skeleton point does not include skeletons corresponding to an upper arm, a forearm, a thigh, and a shank, or the skeleton type indicated by the key skeleton point does not include a head skeleton, or a quantity of skeletons that correspond to an upper arm, a forearm, a thigh, and a shank and that are indicated by the key skeleton point is less than or equal to 3, or the like. Specific content of the second preset condition is not limited herein.

It should be noted that the step 404 and the step 406 are not subject to an execution sequence.

It should be noted that the step 402 and the step 403 and the step 404 to the step 406 are not subject to an execution sequence, and the step 402 and the step 403 and the step 404 to the step 406 may be simultaneously performed, or the step 402 and the step 403 may be performed before the step 404 to the step 406, or the step 404 to the step 406 may be performed before the step 402 and the step 403.

407: Obtain three-dimensional key skeleton point information based on the pose of the camera and the three-dimensional point cloud information and according to an impact detection algorithm.

Converted 3D key skeleton point coordinates corresponding to the 2D key skeleton point are obtained according to an impact detection (HitTest) algorithm and based on the pose of the camera obtained in the step 402 and the three-dimensional point cloud information obtained in the step 403. The three-dimensional key skeleton point information includes three-dimensional coordinates of the key skeleton point and an identifier of the key skeleton point corresponding to the three-dimensional coordinates.

For a principle of an impact detection (HitTest) algorithm, refer to a SLAM technology. FIG. 5 is a schematic diagram of converting a two-dimensional key skeleton point into a three-dimensional key skeleton point according to an embodiment of this application. An optical center of the camera is used as a start point to emit a virtual ray to the detected 2D key skeleton point based on the pose of the camera and the 3D point cloud information. HitTest is performed on the ray and the 3D point cloud to obtain the converted 3D key skeleton point coordinates corresponding to the 2D key skeleton point. A further method for impact detection is the conventional technology. Details are not described herein. It should be noted that, when the ray collides with the 3D point cloud, if no impact result is obtained for the ray, a search may be performed within a specific range around the ray, and a final impact result is obtained by neighbor 3D point interpolation. Finally, the 3D key skeleton point information corresponding to the 2D key skeleton point is output.

Optionally, the pixel coordinates of the two-dimensional key skeleton point in the image are directly converted into three-dimensional coordinates in a world coordinate system based on the image of the object and the pose that is of the camera and that corresponds to the image. The three-dimensional key skeleton point information includes the three-dimensional coordinates of the key skeleton point and the identifier of the key skeleton point.

The three-dimensional point cloud information is obtained by using the computer stereo vision imaging method and based on a plurality of images of the object, or obtained by using the lidar depth imaging method and the like. Therefore, accuracy of the three-dimensional coordinates of the key skeleton point that are obtained according to the impact detection algorithm and based on the pose of the camera and the three-dimensional point cloud information is higher than that of three-dimensional coordinates obtained by directly converting the two-dimensional coordinates of the key skeleton point based on the pose of the camera. It may be understood that a denser three-dimensional point cloud indicates more accurate obtained three-dimensional coordinates of the key skeleton point.

408: Obtain skeleton length information based on the three-dimensional key skeleton point information.

Skeleton length information is obtained based on the three-dimensional key skeleton point information. The skeleton length information includes an identifier of a skeleton and a skeleton length.

Further, every two key skeleton points are connected to form one skeleton. An actual length of each skeleton is obtained based on a three-dimensional space Euclidean distance between 3D joints. The identifier of the skeleton may be determined based on the identifier of the key skeleton point. The identifier of the skeleton indicates the skeleton type. For example, a length of a left thigh skeleton may be obtained based on three-dimensional coordinates of a left hip joint and three-dimensional coordinates of a left knee joint. A length of a left shank skeleton may be obtained based on the three-dimensional coordinates of the left knee joint and three-dimensional coordinates of a left ankle joint. It should be noted that, because a quantity of detected key skeleton points may be missing, the skeleton length information obtained based on the three-dimensional key skeleton point information may include length information of only one skeleton, or include length information of a plurality of skeletons. This is not further limited herein.

Optionally, if the skeleton length information meets a first preset condition, the skeleton length information is deleted. For example, if the first preset condition is that a skeleton length exceeds a preset threshold range, corresponding skeleton length information is deleted. It may be understood that threshold ranges of skeleton lengths of different types of skeletons are different. For example, a range of a skeleton length of a thigh skeleton is different from that of a forearm. In addition, based on a specific type of the measured object, for example, an adult, a child, or another vertebrate other than a person, threshold ranges of skeleton lengths of different types of measured objects may be flexibly set based on statistical information. Alternatively, the first preset condition may be that a skeleton length difference between symmetric parts is greater than or equal to a preset threshold range. For example, if a ratio of a length of a left arm skeleton to a length of a right arm skeleton is greater than or equal to 2 or less than or equal to 0.5, skeleton length information corresponding to the arm is deleted.

409: Obtain posture information of the object based on the three-dimensional key skeleton point information.

A posture of a human body is estimated based on valid skeleton length information obtained in the step 408, to determine the posture information of the object. The posture information may be obtained according to an RMPE algorithm, an instance segmentation (Mask RCNN) algorithm, or the like. This is not limited in this application. The posture information may indicate the posture of the human body, and distinguish a standing posture, a sitting posture, a lying posture, or the like.

If some data in the skeleton length information is missing, the posture information is an incomplete posture. A possible cause is that a part of torso in the image of the object is blocked, some data in the skeleton length information is deleted, or the like.

It should be noted that the step 408 and the step 409 are not subject to an execution sequence.

410: Determine height data of the object based on the posture information and the skeleton length information.

A preset weight parameter is determined based on the posture information of the object in the step 409, and weighted calculation is performed based on the weight parameter and the skeleton length information, to determine the height data of the object.

Optionally, if the posture information of the object is a complete posture, that is, all skeleton length information is valid, weighted height calculation for a height is performed according to Formula (1):

H=Σᵢ₌₁ⁿ(αᵢ*Lᵢ)+β (1)

In Formula (1), n is a quantity of valid skeletons, Lᵢ is a length of an i^{th} skeleton, αᵢ is a weighting coefficient of a length of the i^{th} skeleton, and β is a compensation parameter. Optionally, weighting coefficients αᵢ of skeletons in different postures may be dynamically adjusted, or weighting coefficients corresponding to all skeletons in different postures may be prestored.

Optionally,

β=L_{f1}+L_{f2}=τ₁*L₁+τ₂(Lₙ₋₁Lₙ) (2)

L_{f1} is a compensation value for a distance between a face and a top of a head. Optionally, a value range of L_{f1} is 2 cm to 3 cm. L_{f2} is a compensation value for a distance between an ankle joint and a sole. Optionally, a value range of L_{f2} is 3 cm to 5 cm. L₁ is a skeleton length corresponding to the head, Lₙ₋₁ is a skeleton length corresponding to a thigh, Lₙ is a skeleton length corresponding to a shank, τ₁ is a compensation factor for the distance between the face and the top of the head, and τ₂ is a compensation factor for the distance between the ankle joint and the sole.

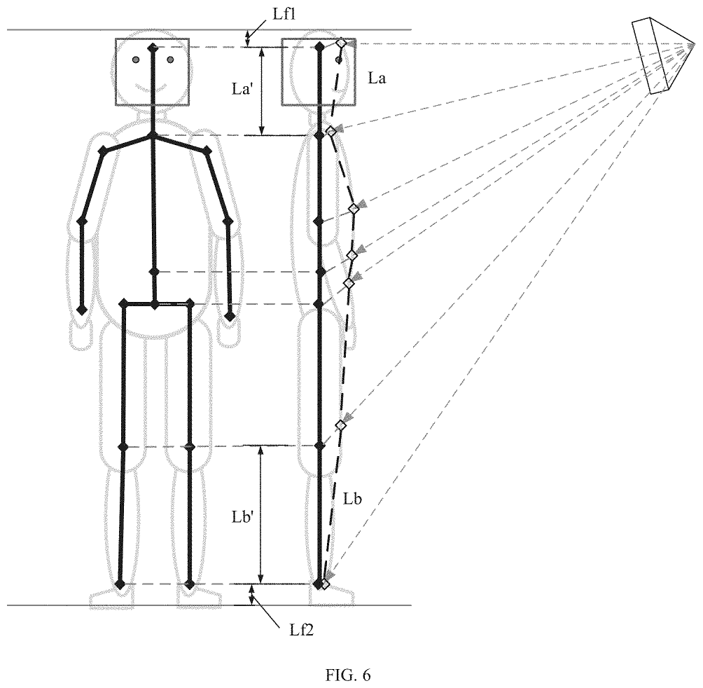

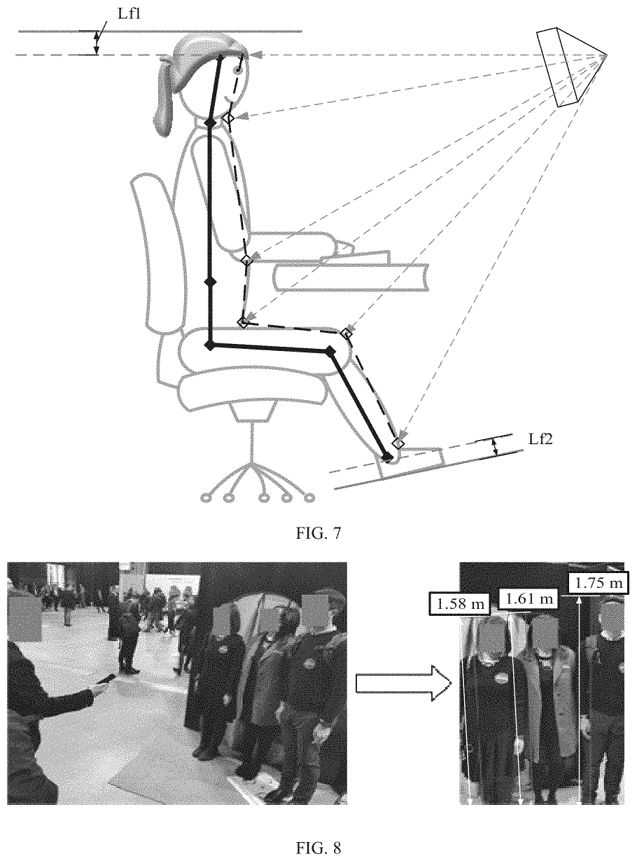

The following briefly describes weighted calculation performed on the skeleton length in height data calculation. For example, FIG. 6 and FIG. 7 are respectively schematic diagrams of height measurement of the standing posture and the sitting posture of the human body.

The skeleton length information obtained based on the three-dimensional key skeleton point information corresponds to a dashed line segment shown in FIG. 6 or FIG. 7. A length of the dashed line segment represents skeleton length information obtained through calculation. Height data expected to be obtained needs to be calculated based on a solid line segment shown in FIG. 6. To implement conversion from the length of the dashed line segment to a length of the solid line segment, calculation is performed based on a preset weighting coefficient in this solution. The length of the solid line segment represents an actual height calculated based on the weighting coefficient and the skeleton length. For example, La is a length corresponding to the head, La′ is an actual head height obtained through weighted calculation, Lb is a length corresponding to the shank, and Lb′ is an actual shank height obtained through weighted calculation.

It should be noted that, for the setting of each weighting coefficient, parameter adjustment is performed based on an empirical value. Alternatively, each weighting coefficient may be trained by using a neural network. Common models include a decision tree, a back propagation (BP) neural network, and the like. This is not limited in this application.

Optionally, when the posture information of the object is an incomplete posture, the weighting coefficient of the skeleton may be adjusted based on the valid skeleton length information, and the height data is calculated. When the obtained valid skeleton length information is incomplete, that is, the posture information of the object is an incomplete posture, there may be one or more pieces of valid skeleton length information. If there is only one piece of valid skeleton length information, a weighting coefficient is determined for the skeleton. If there are a plurality of pieces of valid skeleton length information, a weighting coefficient is determined for each piece of valid skeleton length information in the plurality of pieces of valid skeleton length information. Values of weighting coefficients corresponding to all valid skeletons may be different. A specific value is not limited herein. It may be understood that an error of height data calculated in the incomplete posture increases. Optionally, when a result is displayed, the user may be prompted with that current posture information is an incomplete posture in a plurality of manners, including screen display, a sound prompt, a vibration prompt, or the like. This is not limited herein.

Optionally, after the height data of the object is obtained, the terminal may output the height data to the user in a plurality of manners, including screen display, a sound prompt, a vibration prompt, or the like. This is not limited herein.

Optionally, as shown in FIG. 8, when the measurement is completed, a measurement result is displayed near the image of the object on a screen through a scale line. If a plurality of to-be-measured objects are measured at the same time, height data of each to-be-measured object may be separately displayed near the to-be-measured object in the image of the object.

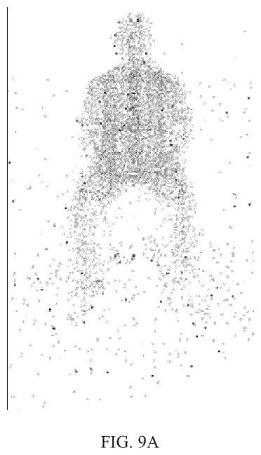

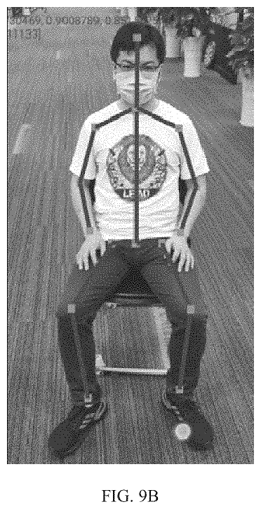

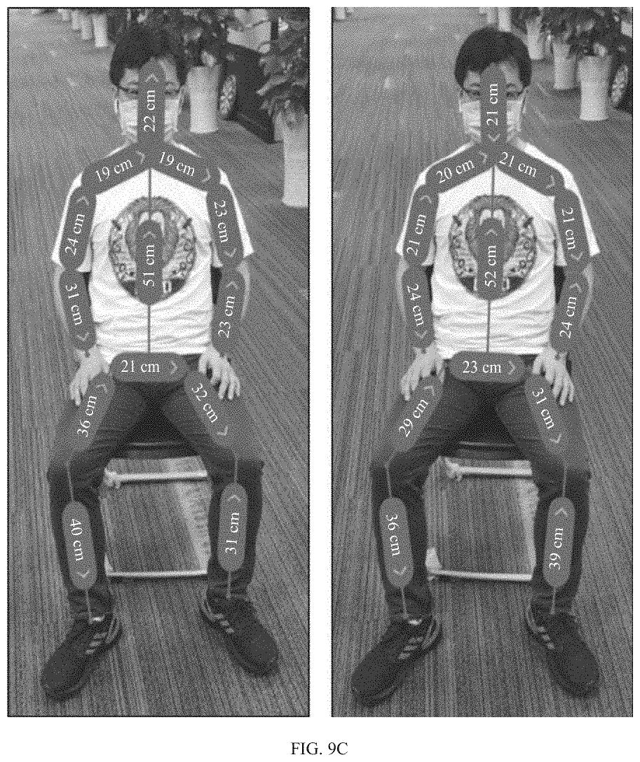

The following describes simulation experiment results of the height measurement method, as shown in FIG. 9A to FIG. 9C.

An ambient environment is scanned. The SLAM system obtains, through calculation, a 3D point cloud corresponding to the measured object. Distribution of the 3D point cloud is shown in FIG. 9A. When the measured object is photographed, a skeleton detection module performs 2D skeleton point detection (in the example, 15 key skeleton points are detected according to the skeleton detection algorithm). A 2D detection result is shown in FIG. 9B. A coordinate conversion module converts 2D coordinates to 3D coordinates, and calculates a length of each 3D skeleton point. Calculation of each skeleton length during actual operation is shown in FIG. 9C. FIG. 9C separately shows results of two measurements. It can be seen that when measurement is performed from different distances and different angles, measured skeleton lengths may fluctuate. In this case, a data integration module is required to perform weighting. The height is finally calculated. The following describes a calculation process. Skeleton lengths obtained by two measurements are shown in Table 2, and the unit is centimeter (cm).

TABLE 2
Example of measured values and weighting coefficients of skeletons

| | Measurement | Measurement | | Converted | Converted |
|---|---|---|---|---|---|
| Skeleton length | 1 | 2 | Weight | value 1 | value 2 |
| Head compensation | | | 0.25 | 5.54 | 5.19 |
| Top of a head to neck | 22.16 | 20.76 | 1.05 | 23.26 | 21.80 |
| Left shoulder to neck | 19.23 | 19.81 | 0.00 | 0.00 | 0.00 |
| Right shoulder to neck | 19.48 | 21.33 | 0.00 | 0.00 | 0.00 |
| Left elbow to left | 23.00 | 20.73 | 0.00 | 0.00 | 0.00 |
| shoulder | | | | | |
| Right elbow to right | 24.02 | 21.38 | 0.00 | 0.00 | 0.00 |
| shoulder | | | | | |
| Left wrist to left elbow | 23.48 | 24.26 | 0.00 | 0.00 | 0.00 |
| Right wrist to right | 31.39 | 24.25 | 0.00 | 0.00 | 0.00 |
| elbow | | | | | |
| Neck to hipbone | 51.16 | 51.95 | 1.15 | 58.84 | 59.74 |
| Left hip to right hip | 20.58 | 22.51 | 0.00 | 0.00 | 0.00 |
| Left knee to left hip | 36.06 | 28.71 | 0.60 | 21.63 | 17.23 |
| Right knee to right hip | 32.27 | 31.16 | 0.60 | 19.36 | 18.70 |
| Left ankle to left knee | 39.91 | 35.58 | 0.55 | 21.95 | 19.57 |
| Right ankle to right | 31.11 | 38.86 | 0.55 | 17.11 | 21.37 |
| knee | | | | | |
| Ankle compensation | | | 0.20 | 8.01 | 7.69 |

| Calculated height | 175.70 | 171.28 |
|---|---|---|
| Actual height | 172 | |
| Calculation error | 3.70 | −0.72 |

The weights and heights are calculated as follows:

(1) During the height measurement, the posture is a normal sitting posture and all skeletons are complete. The skeleton lengths corresponding to the left/right shoulder, the left/right elbow, the left/right wrist, and the hipbone are not used for height calculation. Therefore, the weight is set to 0.

(2) The skeleton lengths from the top of the head to the neck, from the neck to the hipbone, from the left/right knee to the left/right hip, and from the left/right ankle to the left/right knee are respectively assigned with different weights due to a perspective relationship (the weights are related to a photographing angle of the camera and a distance between the camera and the measured object, and a detailed calculation process is not described herein).

(3) A head compensation value is obtained by weighting based on the skeleton length from the top of the head to the neck. An ankle compensation value is obtained by weighting based on an average skeleton length of the skeleton lengths from the left/right hip to the left/right knee to the left/right ankle.

(4) In the example, the actual height of the measured object is 172 cm. In this method, heights calculated after two measurements are weighted are respectively 175.7 cm and 171.3 cm, error percentages are respectively 2.15% and −0.42%, and an average measurement error is 1.28%.

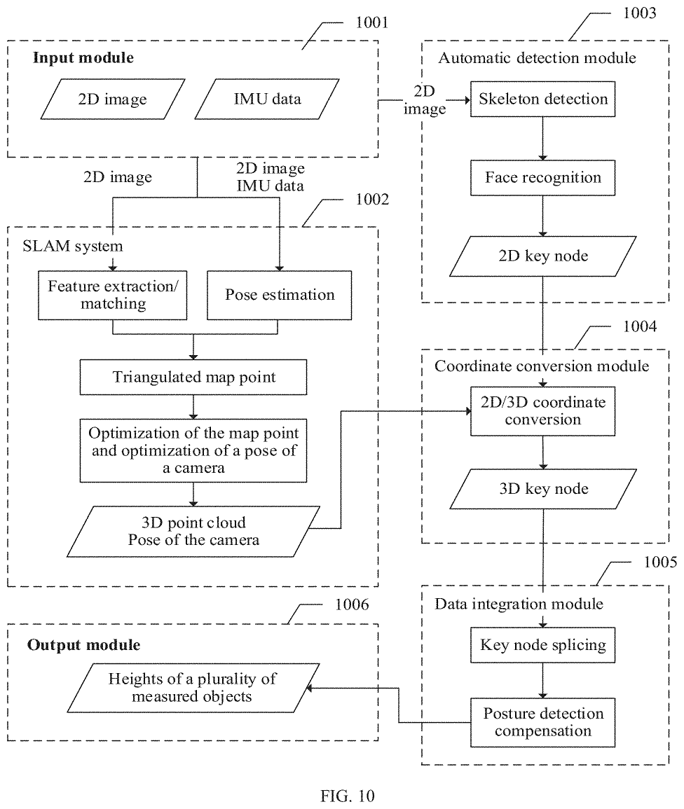

The foregoing describes the height measurement method provided in this application. The following describes a terminal that implements the height measurement method. FIG. 10 is a schematic diagram of an embodiment of a terminal according to an embodiment of this application.

The terminal in this embodiment of this application may be various types of terminal devices such as a mobile phone, a tablet computer, a notebook computer, or a wearable portable device. This is not specifically limited.

The terminal includes the following modules: an input module 1001, a SLAM system 1002, an automatic detection module 1003, a coordinate conversion module 1004, a data integration module 1005, and an output module 1006.

The input module 1001 obtains a real-time 2D image and IMU data.

The SLAM system 1002 may perform pose estimation based on the 2D image and the IMU data to obtain a corresponding pose of a camera when the 2D image is photographed. In addition, processing such as feature extraction, feature matching, and outlier elimination is performed on the 2D image to output feature matching pairs between images. Based on a pose estimation result, a 3D point cloud generation module (corresponding to a triangulated map point in FIG. 10) calculates, according to an algorithm such as triangulation, a 3D point corresponding to a 2D feature point based on the feature matching pairs between images and the pose of the camera that is obtained through estimation. An optimization module (corresponding to map point optimization and camera pose optimization in FIG. 6) inputs the pose of the camera and 3D point cloud data, to perform joint optimization on the pose of the camera and a 3D point cloud. After the foregoing steps are performed, the SLAM system 1002 outputs a real-time pose of the camera and the 3D point cloud data to another module for use. A specific algorithm of the SLAM system may be any one in the conventional technology. This is not limited in this application.

The automatic detection module 1003 detects, based on real-time image data, a 2D key joint (that is, a 2D key skeleton point) of each object according to algorithms such as human body segmentation, skeleton detection, and face detection.

The coordinate conversion module 1004 converts the 2D key joint into a 3D key joint (that is, a 3D key skeleton point) based on the pose of the camera and the 3D point cloud data.

The data integration module 1005 performs key joint splicing based on information about the 3D key joint to obtain torso information of a measured object, and inputs the 3D torso information into a posture detection module for posture detection. A compensation module superimposes corresponding compensation based on different detected postures. A measurement result of a measured user is finally obtained.

The output module 1006 outputs height information of a plurality of measured objects.

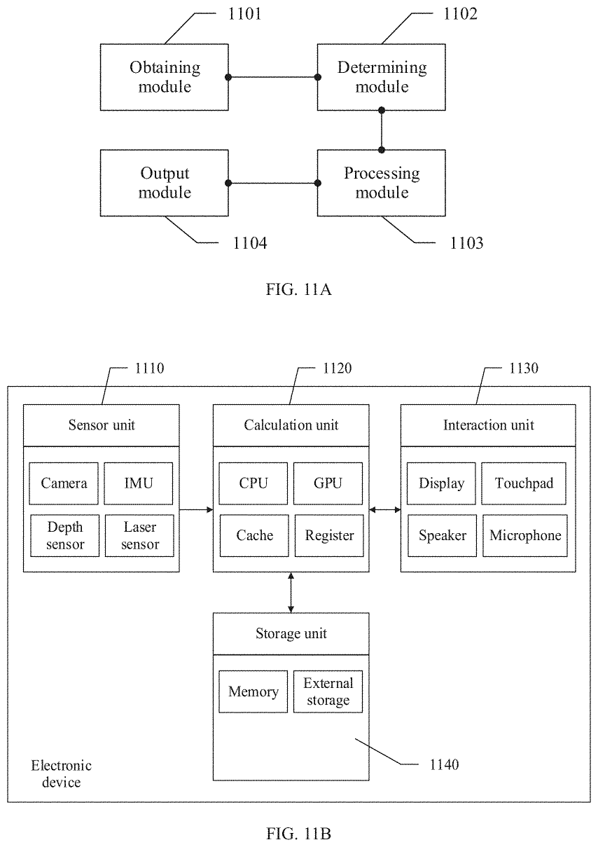

FIG. 11A is a schematic diagram of another embodiment of a terminal according to an embodiment of this application.

The terminal includes an obtaining module 1101 and a determining module 1102.

The obtaining module 1101 is configured to obtain an image including a target object and a pose of a camera used when the image is photographed.

The obtaining module 1101 is further configured to obtain pixel coordinates of at least two key skeleton points of the target object in the image. The pixel coordinates indicate two-dimensional location information of the key skeleton points in the image.

The obtaining module 1101 is further configured to obtain three-dimensional coordinates of the key skeleton points based on the pose of the camera and the pixel coordinates of the key skeleton points. The three-dimensional coordinates indicate three-dimensional location information of the key skeleton points in a world coordinate system. The three-dimensional coordinates of the at least two key skeleton points indicate information about a distance between the at least two key skeleton points.

The determining module 1102 is configured to determine height data of the target object based on the three-dimensional coordinates of the at least two key skeleton points.

Optionally, the obtaining module 1101 is further configured to obtain three-dimensional point cloud information of the target object.

Obtaining the three-dimensional coordinates of the key skeleton points of the target object based on the pose of the camera and the pixel coordinates of the key skeleton points further includes obtaining, based on the pixel coordinates of the key skeleton points, the pose of the camera, and the three-dimensional point cloud information, the three-dimensional coordinates of the key skeleton points according to an impact detection algorithm.

Optionally, the obtaining module 1101 is further configured to obtain the three-dimensional point cloud information of the target object based on at least two images of the target object photographed from different orientations.

Optionally, the obtaining module 1101 is further configured to obtain the three-dimensional point cloud information that is of the target object and that is collected by a depth sensor. The depth sensor includes a binocular camera, a laser radar, a millimeter-wave radar, or a time of flight sensor.

Optionally, the obtaining module 1101 is further configured to obtain the at least two images of the target object photographed from different orientations, where the at least two images of the target object photographed from different orientations include the image; and obtain the pose of the camera based on the at least two images of the target object photographed from different orientations.

Optionally, the obtaining module 1101 is further configured to obtain the at least two images of the target object photographed from different orientations, where the at least two images of the target object photographed from different orientations include the image of the target object; obtain inertial measurement unit data that is of the camera and that corresponds to the at least two images of the target object photographed from different orientations; and determine the pose of the camera based on the inertial measurement unit data and the at least two images of the target object photographed from different orientations.

Optionally, the determining module 1102 is further configured to obtain a skeleton length of the target object and posture information of the target object based on the three-dimensional coordinates of the key skeleton points; determine a preset weight parameter of the skeleton length based on the posture information; and determine the height data of the target object based on the skeleton length and the weight parameter.

Optionally, the skeleton length includes a skeleton length of a head and a skeleton length of a leg.

The determining module 1102 is further configured to determine a head height compensation value based on the skeleton length of the head and a preset head compensation parameter; determine a foot height compensation value based on the skeleton length of the leg and a preset foot compensation parameter; and determine the height data of the target object based on the skeleton length information, the weight parameter, the head height compensation value, and the foot height compensation value.

Optionally, the image includes at least two target objects.

The apparatus further includes a processing module 1103 configured to perform face detection on the image, and determine pixel coordinates of a key skeleton point of each of the at least two target objects from the pixel coordinates of the key skeleton points according to an image segmentation algorithm.

Optionally, the apparatus further includes an output module 1104 configured to display information about the at least two target objects to a user, where the information about the at least two target objects includes at least one of image information of the at least two target objects, image information marked with pixel coordinates of key skeleton points of the at least two target objects, and face detection result information of the at least two target objects.

The obtaining module 1101 is further configured to obtain a user instruction. The user instruction instructs to perform height measurement on one or more of the at least two target objects.

Optionally, the determining module 1102 is further configured to obtain skeleton length information of the target object based on the three-dimensional coordinates of the key skeleton points; delete skeleton length information that meets a first preset condition, where the first preset condition includes skeleton length information in which a skeleton length falls outside a preset range, or a skeleton length difference between symmetric parts being greater than or equal to a preset threshold range; and determine the height data of the target object based on skeleton length information obtained after deletion.

Optionally, the apparatus further includes an output module 1104 configured to label the height data of the target object near the target object in the image, and display the height data to the user; or broadcast the height data of the target object through voice.

Optionally, the apparatus further includes an output module 1104 configured to: if the key skeleton point of the target object does not meet a second preset condition, display detection failure information to the user, or prompt the user with the detection failure information through voice, or prompt the user with the detection failure information through vibration.

The terminal provided in this embodiment of this application may be used to detect a height. The obtaining module is used to obtain the pixel coordinates of the key skeleton points of the target object in the image and obtain the three-dimensional coordinates of the key skeleton points in three-dimensional space. The determining module may determine the height data of the target object based on the three-dimensional coordinates of the at least two key skeleton points. The apparatus converts the two-dimensional pixel coordinates of the key skeleton points into the three-dimensional coordinates, and directly obtains the height data of the target object without conversion of a reference object. This can avoid a measurement error caused by conversion of the reference object when a scenario around the target object is complex, and can improve accuracy of a height measurement result.

FIG. 11B is a schematic diagram of still another embodiment of a terminal according to an embodiment of this application.

The terminal in this application includes a sensor unit 1110, a calculation unit 1120, a storage unit 1140, and an interaction unit 1130.

The sensor unit 1110 generally includes a visual sensor (for example, a camera), configured to obtain 2D image information of a scenario; an IMU configured to obtain motion information of the terminal, such as a linear acceleration and an angular velocity; and a depth sensor/laser sensor (optional) configured to obtain depth information of the scenario.

The calculation unit 1120 usually includes a central processing unit (CPU), a graphics processing unit (GPU), a cache, a register, and the like, and is mainly configured to run an operating system, and process algorithm modules in this application, such as a SLAM system, skeleton detection, and face recognition.

The storage unit 1140 mainly includes a memory and an external storage, and is mainly configured to read and write local and temporary data of a user.

The interaction unit 1130 mainly includes a display, a touchpad, a speaker, a microphone, and the like, and is mainly configured to interact with the user, obtain an input, and implement display of algorithm effect and the like.

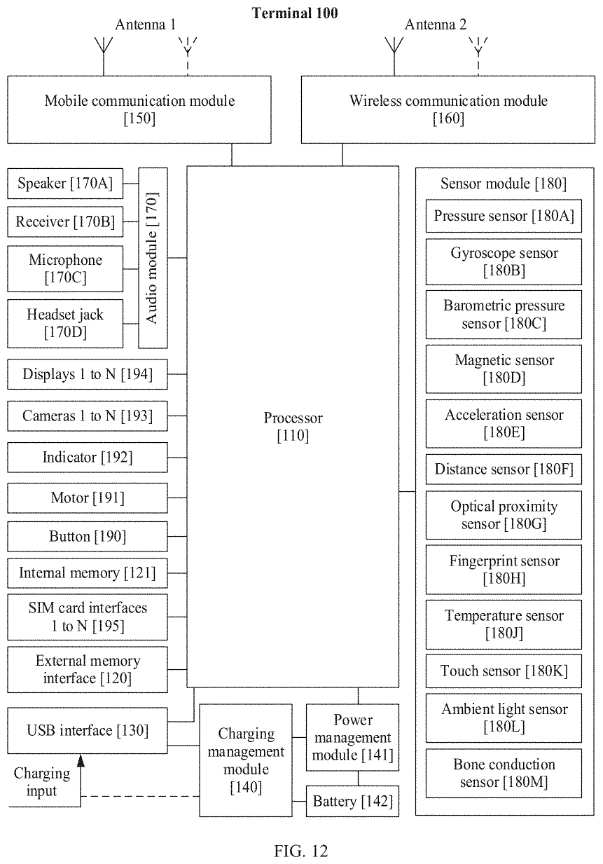

FIG. 12 is a schematic diagram of an embodiment of a terminal according to an embodiment of this application.

For ease of understanding, the following describes, by using an example, a structure of a terminal 100 provided in an embodiment of this application. FIG. 13 is a schematic diagram of a structure of a terminal according to an embodiment of this application.

As shown in FIG. 13, the terminal 100 may include a processor 110, an external memory interface 120, an internal memory 121, a Universal Serial Bus (USB) interface 130, a charging management module 140, a power management module 141, a battery 142, an antenna 1, an antenna 2, a mobile communication module 150, a wireless communication module 160, an audio module 170, a speaker 170A, a receiver 170B, a microphone 170C, a headset jack 170D, a sensor module 180, a button 190, a motor 191, an indicator 192, a camera 193, a display 194, a subscriber identity module (SIM) card interface 195, and the like. The sensor module 180 may include a pressure sensor 180A, a gyroscope sensor 180B, a barometric pressure sensor 180C, a magnetic sensor 180D, an acceleration sensor 180E, a distance sensor 180F, an optical proximity sensor 180G, a fingerprint sensor 180H, a temperature sensor 180J, a touch sensor 180K, an ambient light sensor 180L, a bone conduction sensor 180M, and the like.

It may be understood that the structure shown in this embodiment of this application does not constitute a specific limitation on the terminal 100. In some other embodiments of this application, the terminal 100 may include more or fewer components than those shown in the figure, or may combine some components, or may split some components, or may have different component arrangements. The components shown in the figure may be implemented by hardware, software, or a combination of software and hardware.

The processor 110 may include one or more processing units. For example, the processor 110 may include an application processor (AP), a modem processor, a GPU, an image signal processor (ISP), a controller, a memory, a video codec, a digital signal processor (DSP), a baseband processor, a neural-network processing unit (NPU), and/or the like. Different processing units may be independent components, or may be integrated into one or more processors.

The controller may be a nerve center and a command center of the terminal 100. The controller may generate an operation control signal based on instruction operation code and a time sequence signal, to complete control of instruction reading and instruction execution.

A memory may be further disposed in the processor 110, and is configured to store instructions and data. In some embodiments, the memory in the processor 110 is a cache memory. The memory may store instructions or data just used or cyclically used by the processor 110. If the processor 110 needs to use the instructions or the data again, the processor may directly invoke the instructions or the data from the memory. This avoids repeated access, reduces waiting time of the processor 110, and improves system efficiency.

In some embodiments, the processor 110 may include one or more interfaces. The interface may include an inter-integrated circuit (I2C) interface, an Inter-Integrated Circuit Sound (I2S) interface, a pulse-code modulation (PCM) interface, a universal asynchronous receiver/transmitter (UART) interface, a mobile industry processor interface (MIPI), a general-purpose input/output (GPIO) interface, a SIM interface, a USB interface, and/or the like.

It may be understood that an interface connection relationship between the modules illustrated in embodiments of this application is merely an example for description, and does not constitute a limitation on a structure of the terminal 100. In some other embodiments of this application, the terminal 100 may alternatively use an interface connection manner different from that in the foregoing embodiment, or use a combination of a plurality of interface connection manners.

The charging management module 140 is configured to receive a charging input from a charger. The charger may be a wireless charger or a wired charger. In some embodiments of wired charging, the charging management module 140 may receive a charging input of the wired charger through the USB interface 130.

The power management module 141 is configured to connect the battery 142 and the charging management module 140 to the processor 110. The power management module 141 receives an input of the battery 142 and/or the charging management module 140 to supply power to the processor 110, the internal memory 121, an external memory, the display 194, the camera 193, the wireless communication module 160, and the like.

A wireless communication function of the terminal 100 may be implemented by using the antenna 1, the antenna 2, the mobile communication module 150, the wireless communication module 160, the modem processor, the baseband processor, and the like.

In some feasible implementations, the terminal 100 may communicate with another device by using a wireless communication function. For example, the terminal 100 may communicate with a second electronic device. The terminal 100 establishes a projection connection to the second electronic device. The terminal 100 outputs projection data to the second electronic device and the like. The projection data output by the terminal 100 may be audio or video data.

The antenna 1 and the antenna 2 are configured to transmit and receive an electromagnetic wave signal. Each antenna in the terminal 100 may be configured to cover one or more communication frequency bands. Different antennas may be further multiplexed, to improve antenna utilization. For example, the antenna 1 may be multiplexed as a diversity antenna of a wireless local area network. In some other embodiments, the antenna may be used in combination with a tuning switch.

The mobile communication module 150 may provide a wireless communication solution that includes first generation (1G)/third generation (3G)/fourth generation (4G)/fifth generation (5G) or the like and that is applied to the terminal 100. The mobile communication module 150 may include at least one filter, a switch, a power amplifier, a low-noise amplifier (LNA), and the like. The mobile communication module 150 may receive an electromagnetic wave through the antenna 1, perform processing such as filtering or amplification on the received electromagnetic wave, and transmit the electromagnetic wave to the modem processor for demodulation. The mobile communication module 150 may further amplify a signal modulated by the modem processor, and convert the signal into an electromagnetic wave for radiation through the antenna 2. In some embodiments, at least some functional modules in the mobile communication module 150 may be disposed in the processor 110. In some embodiments, at least some functional modules of the mobile communication module 150 and at least some modules of the processor 110 may be disposed in a same component.

The modem processor may include a modulator and a demodulator. The modulator is configured to modulate a to-be-sent low-frequency baseband signal into a medium-high frequency signal. The demodulator is configured to demodulate a received electromagnetic wave signal into a low-frequency baseband signal. Then, the demodulator transmits the low-frequency baseband signal obtained through demodulation to the baseband processor for processing. The low-frequency baseband signal is processed by the baseband processor and then transmitted to the application processor. The application processor outputs a sound signal by an audio device (which is not limited to the speaker 170A, the receiver 170B, or the like), or displays an image or a video on the display 194. In some embodiments, the modem processor may be an independent component. In some other embodiments, the modem processor may be independent of the processor 110, and is disposed in a same device as the mobile communication module 150 or another functional module.

The wireless communication module 160 may provide a wireless communication solution that is applied to the terminal 100, and that includes a wireless local area network (WLAN) (for example, a Wi-Fi network), BLUETOOTH (BT), a global navigation satellite system (GNSS), frequency modulation (FM), a near-field communication (NFC) technology, an infrared (IR) technology, and the like. The wireless communication module 160 may be one or more components integrating at least one communication processing module. The wireless communication module 160 receives an electromagnetic wave through the antenna 1, performs frequency modulation and filtering processing on an electromagnetic wave signal, and sends a processed signal to the processor 110. The wireless communication module 160 may further receive a to-be-sent signal from the processor 110, perform frequency modulation and amplification on the signal, and convert the signal into an electromagnetic wave for radiation through the antenna 2.

In some embodiments, the antenna 1 of the terminal 100 is coupled to the mobile communication module 150, and the antenna 2 is coupled to the wireless communication module 160, so that the terminal 100 may communicate with a network and another device by using a wireless communication technology. The wireless communication technology may include a Global System for Mobile Communications (GSM), a General Packet Radio Service (GPRS), code-division multiple access (CDMA), wideband CDMA (WCDMA), time-division CDMA (TD-SCDMA), Long-Term Evolution (LTE), BT, a GNSS, a WLAN, NFC, FM, an IR technology, and/or the like. The GNSS may include a Global Positioning System (GPS), a global navigation satellite system (GLONASS), a BEIDOU navigation satellite system (BDS), a quasi-zenith satellite system (QZSS), and/or a satellite based augmentation system (SBAS).

The terminal 100 implements the display function through the GPU, the display 194, the application processor, and the like. The GPU is a microprocessor for image processing, and is connected to the display 194 and the application processor. The GPU is configured to perform mathematical and geometric computation, and render an image. The processor 110 may include one or more GPUs that execute program instructions to generate or change display information.

The display 194 is configured to display an image, a video, and the like. The display 194 includes a display panel. The display panel may be a liquid-crystal display (LCD), an organic light-emitting diode (LED) (OLED), an active-matrix OLED (AMOLED), a flexible LED (FLED), a mini-LED, a micro-LED, a micro-OLED, a quantum dot LED (QLED), or the like. In some embodiments, the terminal 100 may include one or N displays 194, where N is a positive integer greater than 1.

In some feasible implementations, the display 194 may be configured to display each interface output by the system of the terminal 100. For each interface output by the terminal 100, refer to related descriptions in the following embodiments.

The terminal 100 may implement a photographing function by using the image signal processing (ISP), the camera 193, the video codec, the GPU, the display 194, the application processor, and the like.

The ISP is configured to process data fed back by the camera 193. For example, during photographing, a shutter is touched, and light is transmitted to a photosensitive element of the camera through a lens. An optical signal is converted into an electrical signal, and the photosensitive element of the camera transmits the electrical signal to the ISP for processing, to convert the electrical signal into a visible image. The ISP may further perform algorithm optimization on noise, brightness, and complexion of the image. The ISP may further optimize parameters such as exposure and a color temperature of a photographing scenario. In some embodiments, the ISP may be disposed in the camera 193.

The camera 193 is configured to capture a static image or a video. An optical image of an object is generated through the lens, and is projected onto the photosensitive element. The photosensitive element may be a charge-coupled device (CCD) or a complementary metal-oxide-semiconductor (CMOS) phototransistor. The photosensitive element converts an optical signal into an electrical signal, and then transmits the electrical signal to the ISP to convert the electrical signal into a digital image signal. The ISP outputs the digital image signal to the DSP for processing. The DSP converts the digital image signal into an image signal in a standard format such as a red, green, and blue (RGB) or a luma, blue projection, red projection (YUV). In some embodiments, the terminal 100 may include one or N cameras 193, where N is a positive integer greater than 1.

The digital signal processor is configured to process a digital signal, and may process another digital signal in addition to the digital image signal.

The video codec is configured to compress or decompress a digital video. The terminal 100 may support one or more video codecs. In this way, the terminal 100 can play or record videos in a plurality of coding formats, for example, Moving Picture Experts Group (MPEG)-1, MPEG-2, MPEG-3, and MPEG-4.

The NPU is a neural-network (NN) computing processor, quickly processes input information by referring to a structure of a biological neural network, for example, by referring to a mode of transfer between human brain neurons, and may further continuously perform self-learning. Applications such as intelligent cognition of the terminal 100 may be implemented by using the NPU, for example, image recognition, facial recognition, speech recognition, and text understanding.

The external memory interface 120 may be configured to connect to an external memory card such as a micro SanDisk (SD) card, to extend a storage capability of the terminal 100. The external memory card communicates with the processor 110 through the external memory interface 120, to implement a data storage function. For example, files such as music and videos are stored in the external memory card.

The internal memory 121 may be configured to store computer-executable program code. The executable program code includes instructions. The processor 110 runs the instructions stored in the internal memory 121, to perform various function applications of the terminal 100 and data processing. The internal memory 121 may include a program storage region and a data storage region. The program storage region may store an operating system, an application required by at least one function (for example, a voice playing function or an image playing function), and the like. The data storage region may store data (such as audio data and a phone book) created during use of the terminal 100, and the like. In addition, the internal memory 121 may include a high-speed random access memory, or may include a nonvolatile memory, for example, at least one magnetic disk storage device, a flash memory, or a universal flash storage (UFS).

The terminal 100 may implement audio functions such as music playing and recording through the audio module 170, the speaker 170A, the receiver 170B, the microphone 170C, the headset jack 170D, the application processor, and the like. In some feasible implementations, the audio module 170 may be configured to play a sound corresponding to a video. For example, when the display 194 displays a video play picture, the audio module 170 outputs a video play sound.

The audio module 170 is configured to convert digital audio information into an analog audio signal for output, and is also configured to convert an analog audio input into a digital audio signal.

The speaker 170A, also referred to as a “horn”, is configured to convert an audio electrical signal into a sound signal.

The receiver 170B, also referred to as an “earpiece”, is configured to convert an audio electrical signal into a sound signal.

The microphone 170C, also referred to as a “mike” or a “mic”, is configured to convert a sound signal into an electrical signal.

The headset jack 170D is configured to connect to a wired headset. The headset jack 170D may be a USB interface 130, or may be a 3.5 millimeter (mm) Open Mobile Terminal Platform (OMTP) standard interface or cellular telecommunications industry association (CTIA) of the United States of America (USA) standard interface.

The pressure sensor 180A is configured to sense a pressure signal, and can convert the pressure signal into an electrical signal. In some embodiments, the pressure sensor 180A may be disposed in the display 194. The gyroscope sensor 180B may be configured to determine a motion posture of the terminal 100. The barometric pressure sensor 180C is configured to measure barometric pressure.

The acceleration sensor 180E may detect magnitudes of accelerations of the terminal 100 in various directions (including three axes or six axes). A magnitude and a direction of gravity may be detected when the terminal 100 is still. The acceleration sensor 180E may be further configured to identify a posture of the terminal, and is used in an application such as a pedometer or screen switching between a landscape mode and a portrait mode.

The distance sensor 180F is configured to measure a distance.

The ambient light sensor 180L is configured to sense ambient light brightness.

The fingerprint sensor 180H is configured to collect a fingerprint.

The temperature sensor 180J is configured to detect a temperature.

The touch sensor 180K is also referred to as a “touch panel”. The touch sensor 180K may be disposed in the display 194, and the touch sensor 180K and the display 194 form a touchscreen, which is also referred to as a “touchscreen”. The touch sensor 180K is configured to detect a touch operation performed on or near the touch sensor. The touch sensor may transfer the detected touch operation to the application processor to determine a type of a touch event. A visual output related to the touch operation may be provided through the display 194. In some other embodiments, the touch sensor 180K may alternatively be disposed on a surface of the terminal 100 at a location different from that of the display 194.

The button 190 includes a power button, a volume button, and the like. The button 190 may be a mechanical button, or may be a touch button. The terminal 100 may receive a button input, and generate a button signal input related to a user setting and function control of the terminal 100.

The motor 191 may generate a vibration prompt.

The indicator 192 may be an indicator light, and may be configured to indicate a charging status and a power change, or may be configured to indicate a message, a missed call, a notification, and the like.

The SIM card interface 195 is configured to connect to a SIM card.

It may be clearly understood by a person skilled in the art that, for ease and brevity of description, for a detailed working process of foregoing systems, apparatuses, and units, refer to a corresponding process in the foregoing method embodiments. Details are not described herein again.

In the several embodiments provided in this application, it should be understood that the disclosed system, apparatus, and method may be implemented in another manner. For example, the described apparatus embodiments are merely examples. For example, division into the units is merely logical function division and may be other division in actual implementation. For example, a plurality of units or components may be combined or integrated into another system, or some features may be ignored or not performed. In addition, the displayed or discussed mutual couplings or direct couplings or communication connections may be implemented through some interfaces. The indirect couplings or communication connections between the apparatuses or units may be implemented in electronic, mechanical, or other forms.

The units described as separate parts may or may not be physically separate, and parts displayed as units may or may not be physical units, and may be located in one location, or may be distributed on a plurality of network units. Some or all of the units may be selected based on actual requirements to achieve the objectives of the solutions of embodiments.

In addition, functional units in embodiments of this application may be integrated into one processing unit, or each of the units may exist alone physically, or two or more units may be integrated into one unit. The integrated unit may be implemented in a form of hardware, or may be implemented in a form of a software functional unit.

When the integrated unit is implemented in the form of the software functional unit and sold or used as an independent product, the integrated unit may be stored in a computer-readable storage medium. Based on such an understanding, the technical solutions of this application essentially, or the part contributing to the conventional technology, or all or some of the technical solutions may be implemented in a form of a software product. The computer software product is stored in a storage medium and includes several instructions for instructing a computer device (which may be a personal computer, a server, or a network device) to perform all or some of the steps of the methods described in embodiments of this application. The foregoing storage medium includes any medium that can store program code, for example, a USB flash drive, a removable hard disk, a read-only memory (ROM), a random-access memory (RAM), a floppy disk, or a compact disc.

The foregoing embodiments are merely intended for describing the technical solutions of this application other than limiting this application. Although this application is described in detail with reference to the foregoing embodiments, a person of ordinary skill in the art should understand that modifications may still be made to the technical solutions described in the foregoing embodiments or equivalent replacements may still be made to some technical features thereof, without departing from the spirit and scope of the technical solutions of embodiments of this application.

What is claimed is:

1. A method comprising:
obtaining an image comprising a target object;
obtaining a pose of a camera when the image is photographed;
obtaining first pixel coordinates of at least two key skeleton points of the target object, wherein each of the at least two key skeleton points comprises a skeleton joint, and wherein the first pixel coordinates indicate two-dimensional location information;
obtaining first three-dimensional coordinates of the at least two key skeleton points based on the pose and the first pixel coordinates, wherein the first three-dimensional coordinates indicate three-dimensional location information and first information about a distance between the at least two key skeleton points; and
determining height data of the target object based on the first three-dimensional coordinates.

2. The method of claim 1, wherein determining the height data comprises:
obtaining second pixel coordinates of at least three key skeleton points of the target object;
obtaining second three-dimensional coordinates of the at least three key skeleton points based on the pose and the second pixel coordinates, wherein the second three-dimensional coordinates indicate second three-dimensional location information, and second information about distances between the at least three key skeleton points;
determining at least two skeleton distances based on the second three-dimensional coordinates; and
determining the height data based on the at least two skeleton distances.

3. The method of claim 1, wherein the coordinate system comprises a world coordinate system.

4. The method of claim 1, further comprising obtaining three-dimensional point cloud information of the target object, wherein obtaining the first three-dimensional coordinates comprises obtaining, based on the first pixel coordinates, the pose, and the three-dimensional point cloud information, the first three-dimensional coordinates according to an impact detection algorithm.

5. The method of claim 4, wherein obtaining the three-dimensional point cloud information comprises obtaining the three-dimensional point cloud information of the target object based on at least two images of the target object photographed from different orientations.

6. The method of claim 4, wherein obtaining the three-dimensional point cloud information comprises obtaining the three-dimensional point cloud information that is of the target object and that is collected by a depth sensor, and wherein the depth sensor comprises a binocular camera, a laser radar, a millimeter-wave radar, or a time of flight sensor.

7. The method of claim 1, wherein obtaining the image comprises:
obtaining at least two images of the target object photographed from different orientations, wherein the at least two images comprise the image; and
obtaining the pose based on the at least two images.

8. The method of claim 1, wherein obtaining the image comprises:
obtaining at least two images of the target object photographed from different orientations, wherein the at least two images comprise the image;
obtaining inertial measurement unit data that is of the camera and that corresponds to the at least two images; and
determining the pose based on the inertial measurement unit data and the at least two images.

9. The method of claim 1, wherein determining the height data comprises:
obtaining a first skeleton length of the target object and posture information of the target object based on the first three-dimensional coordinates;
determining a preset weight parameter of the skeleton length based on the posture information; and
determining the height data based on the first skeleton length and the preset weight parameter.

10. The method of claim 9, wherein the first skeleton length comprises a second skeleton length of a head and a third skeleton length of a leg, and wherein determining the height data comprises:
determining a head height compensation value based on the second skeleton length and a preset head compensation parameter;
determining a foot height compensation value based on the third skeleton length and a preset foot compensation parameter; and
determining the height data based on the first skeleton length, the preset weight parameter, the head height compensation value, and the foot height compensation value.

11. The method of claim 1, wherein the image comprises at least two target objects, and wherein the method further comprises:
performing face detection on the image; and
determining second pixel coordinates of a key skeleton point of each of the at least two target objects from the first pixel coordinates according to an image segmentation algorithm.

12. The method of claim 1, wherein the key skeleton points are arranged in a direction of gravity.

13. The method of claim 1, wherein the target object is in a non-standing posture.

14. The method of claim 1, wherein determining the height data comprises:
obtaining first skeleton length information of the target object based on the first three-dimensional coordinates;
deleting second skeleton length information that meets a first preset condition, wherein the first preset condition comprises third skeleton length information in which a skeleton length falls outside a preset range or comprises a skeleton length difference between symmetric parts being greater than or equal to a preset threshold range; and
determining the height data based on second skeleton length information.

15. The method of claim 1, further comprising:
labeling the height data near the target object in the image, and displaying the height data to a user; or
broadcasting the height data through voice.

16. The method of claim 1, wherein when the at least two key skeleton points of the target object do not meet a first preset condition, the method further comprises:
displaying detection failure information to a user;
prompting the user with the detection failure information through voice; or
prompting the user with the detection failure information through vibration.

17. An apparatus comprising:
a memory configured to store instructions; and
a processor coupled to the memory and configured to execute the instructions to cause the apparatus to:
obtain an image comprising a target object; obtain a pose of a camera when the image is photographed;
obtain first pixel coordinates of at least two key skeleton points of the target object, wherein each of the at least two key skeleton points comprises a skeleton joint, and wherein the first pixel coordinates indicate two-dimensional location information;
obtain first three-dimensional coordinates of the at least two key skeleton points based on the pose and the first pixel coordinates, wherein the first three-dimensional coordinates indicate three-dimensional location information, and first information about a distance between the at least two key skeleton points; and
determine height data of the target object based on the first three-dimensional coordinates.

18. The apparatus of claim 17, wherein the processor is further configured to execute the instructions to cause the apparatus to:
obtain second pixel coordinates of at least three key skeleton points of the target object;
obtain second three-dimensional coordinates of the at least three key skeleton points based on the pose and the second pixel coordinates, wherein the second three-dimensional coordinates indicate second three-dimensional location information, and second information about distances between the at least three key skeleton points;
determine at least two skeleton distances based on the second three-dimensional coordinates; and
determine the height data based on the at least two skeleton distances.

19. The apparatus of claim 17, wherein the processor is further configured to execute the instructions to cause the apparatus to:
obtain three-dimensional point cloud information of the target object; and
obtain, based on the first pixel coordinates, the pose, and the three-dimensional point cloud information, the first three-dimensional coordinates according to an impact detection algorithm.

20. A computer program product comprising computer-executable instructions for storage on a non-transitory computer-readable storage medium that, when executed by a processor, cause an apparatus to:
obtain an image comprising a target object;
obtain a pose of a camera when the image is photographed;
obtain pixel coordinates of at least two key skeleton points of the target object, wherein each of the at least two key skeleton points comprises a skeleton joint, and wherein the pixel coordinates indicate two-dimensional location information;
obtain three-dimensional coordinates of the at least two key skeleton points based on the pose and the pixel coordinates, wherein the three-dimensional coordinates indicate three-dimensional location information, and information about a distance between the at least two key skeleton points; and
determine height data of the target object based on the three-dimensional coordinates.
